(19) United States
(12) Patent Application Publication
(10) Pub. No.: US 2004/0242459 A1
(43) Pub. Date: Dec. 2, 2004

(54) Method for treating inflammatory diseases by administering a ppar-delta agonist

(76) Inventors: Michael J Forrest, Shrewsbury, NJ (US); Joel P Berger, Hoboken, NJ (US); David E Moller, Bedminister, NJ (US); Samuel Wright, Westfield, NJ (US)

Correspondence Address: MERCK AND CO INC, P O BOX 2000, RAHWAY, NJ, 070650907

(21) Appl. No.: 10/480,363

(22) PCT Filed: Jun. 7, 2002
(86) PCT No.: PCT/US02/20974

Related U.S. Application Data
(60) Provisional application No. 60/297,356, filed on Jun. 11, 2001.

(51) Int. Cl. A61K 38/17

(52) U.S. Cl. 514/2

(57) ABSTRACT

A method for treating, controlling, preventing or reducing the risk of contracting an inflammatory disease or condition in a mammalian patient, comprises the steps of (1) selecting a patient in need thereof, and (2) treating the patient with a therapeutically effective amount of a composition comprising a PPAR-δ agonist. Inflammatory diseases that may be treated by this method include but are not limited to rheumatoid arthritis, juvenile rheumatoid arthritis, systemic lupus erythematosus, osteoarthritis, degenerative joint disease, one or more connective tissue diseases, ankylosing spondylitis, and bursitis.

### FIELD OF THE INVENTION

This invention relates to methods for treating inflammatory diseases, such as osteoarthritis and rheumatoid arthritis, by administration of a PPAR delta agonist.

### BACKGROUND OF THE INVENTION

Non-steroidal anti-inflammatory drugs (NSAIDs) are widely used to treat inflammation. These exert anti-inflammatory, analgesic and antipyretic activity. NSAID's include salicylates such as aspirin, sodium salicylate, choline salicylate, salicylsalicylic acid, diflunisal, and salsalate; indoleacetic acids such as indomethacin and sulindac; pyrazoles such as phenylbutazone, oxyphenbutazone; pyrrolealkanoic acids such as tolmetin; phenylacetic acids such as ibuprofen, feroprofen, flurbiprofen, and ketoprofen; fenamates such as mefanamic acid, and meclofenamate; oxicams such as piroxicam; and naphthaleneacetic acids such as naproxen. Nearly all NSAIDs act by inhibiting the activity of the cyclooxygenase-2 enzyme (COX-2). Aspirin, for example, acetylates and irreversibly inactivates cyclooxygenase. Others, such as indomethacin, inhibit cyclooxygenase activity reversibly by binding in a stereospecific manner to one or another of the subunits of the enzyme. NSAIDs are active in reducing the prostaglandin-induced pain and swelling associated with the inflammation process because they inhibit the cyclooxygenase-2 (COX-2) enzyme. Most NSAIDs do not selectively inhibit only the COX-2 enzyme, but also inhibit the cyclooxygenase-1 (COX-1) enzyme, which is important in other biologically beneficial prostaglandin-regulated processes that are not associated with the inflammation process. The use of high doses of non-selective NSAIDs can produce severe side effects, including life threatening ulcers, that limit their therapeutic potential. The new class of selective COX-2 inhibitors, such as rofecoxib, etoricoxib, celecoxib, parecoxib and valdecoxib, generally do not have the same side effects that are exhibited by the earlier NSAIDs.

Adrenal corticosteroids, which are alternatives to NSAIDs for treating inflammatory diseases, also have potentially severe side effects, especially when long term therapy is involved. These steroids, including hydrocortisone, prednisolone, 6-alpha-methylprednisolone, triamcinolone, dexamethasone and betaroethasone, affect inflammation by a possible mechanism whereby they bind to intracellular glucocorticoid receptors to subsequently initiate a series of cellular events involving synthesis of new phospholipid inhibitory proteins, or lipocortins, that can affect the inflammatory and the teratogenic responses of certain cells exposed to glucocorticoids. The anti-inflammatory effect of glucocorticoids has been well documented.

Peroxisome proliferator-activated receptors (PPAR) are members of the nuclear hormone receptor superfamily of ligand-activated transcriptional factors, a large and diverse group of proteins that mediate ligand-dependent transcriptional activation and repression. Three PPAR subtypes have been isolated: PPAR-α, PPAR-δ and PPAR-γ. PPAR-α is found in the liver, heart, kidney, muscle, brown adipose tissue and gut. PPAR-δ, sometimes also known as PPAR-β or NUC1, is ubiquitously expressed. PPAR-γ is expressed in adipose tissue and is thought to regulate differentiation of adipocytes. The PPAR sub-types and ligands have been intensively studied recently because of their usefulness in the treatment of insulin resistance and non-insulin dependent diabetes mellitus (NIDDM), as well as related disorders. PPAR-α agonists, such as fenofibrate, are effective in the treatment of lipid disorders, such as hyperlipidemia and dyslipidemia. PPAR-γ agonists, such as pioglitazone and rosiglitazone, are currently marketed for the treatment of NIDDM because of their beneficial effects on insulin resistance. PPAR-δ is less understood than PPAR-α and PPAR-γ. Recently PPAR-δ agonists have been identified as useful in the treatment of diabetes and related lipod disorders.

The effects of PPAR-α and -γ agonists on inflammation have been investigated, and the data indicate that PPAR-α and -γ agonists reduce certain kinds of inflammation. There are no publications on whether PPAR-α and PPAR-γ agonists have activity in treating inflammation of the joints, osteoarthritis, or rheumatoid arthritis. Whether they have utility in the treatment of joint inflammation and arthritis is therefore still unknown.

Although PPAR-δ has been implicated in the regulation of several biological systems (oligodendrocyte differentiation, colon epithelial cell proliferation, uterine implantation of blastocysts), there are no published data suggesting that PPAR-δ activation may have a role in the treatment, prevention or control of arthritis, joint inflammation or other related inflammatory conditions. As described below, it has now been found that PPAR-δ agonists are in fact useful for treating or inhibiting inflammation, particularly inflammation of the joints and connective tissue, as occurs in numerous diseases and conditions, such as rheumatoid arthritis, related autoimmune diseases, and osteoarthritis.

### SUMMARY OF THE INVENTION

The present invention is a method for treating an inflammatory disease in a mammalian patient, where the method comprises the step of treating the patient with a PPAR-δ agonist. Such diseases include, but are not limited to, rheumatoid arthritis, juvenile rheumatoid arthritis, systemic lupus erythematosus, osteoarthritis, degenerative joint disease, one or more connective tissue diseases, ankylosing spondylitis, bursitis, Sjogren's syndrome, psoriasis, psoriatic arthritis, neuralgia, synovitis, glomerulonephritis, vasculitis, sacoidosis, inflammations that occur as sequellae to influenza, the common cold and other viral infections, gout, contact dermatitis, low back and neck pain, dysmenorrhea, headache, toothache, sprains, strains, myositis, burns, injuries, and pain and inflammation that follow surgical and dental procedures in a patient. Diseases that are likely to be responsive to this treatment include rheumatoid arthritis, juvenile rheumatoid arthritis, systemic lupus erythematosus, osteoarthritis, degenerative joint disease, one or more connective tissue diseases, ankylosing spondylitis, bursitis, Sjogren's syndrome, psoriasis, psoriatic arthritis, neuralgia, synovitis, glomerulonephritis, vasculitis, sacoidosis, inflammations that occur as sequellae to influenza, the common cold and other viral infections, gout, contact dermatitis, low back and neck pain, dysmenorrhea, headache, toothache, sprains, strains, myositis, burns, injuries, and pain and inflammation that follow surgical and dental procedures in a patient.

The invention also is a method for treating an inflammatory disease in a patient which comprises treating the patient with a combination comprising a PPAR-δ agonist and an NSAID, which may be non-selective or may be a selective cyclooxygenase-2 (COX-2) inhibitor. Such diseases include, but are not limited to, the diseases listed above.

The invention is also a method for inhibiting or preventing secondary inflammation in a patient developing inflammation at a primary site which comprises treating the patient with a composition which comprises a PPAR-δ agonist.

### DETAILED DESCRIPTION OF THE INVENTION

The present invention is a method for relieving inflammation and the symptoms of inflammation, including pain, fever, swelling, edema, and redness, that are associated with a variety of diseases and conditions, including, rheumatoid arthritis, juvenile rheumatoid arthritis, systemic lupus erythematosus, osteoarthritis, degenerative joint disease, one or more connective tissue diseases, ankylosing spondylitis, bursitis, Sjogren's syndrome, psoriasis, psoriatic arthritis, neuralgia, synovitis, glomerulonephritis, vasculitis, sacoidosis, inflammations that occur as sequellae to influenza, the common cold and other viral infections, gout, contact dermatitis, low back and neck pain, dysmenorrhea, headache, toothache, sprains, strains, myositis, burns, injuries, and pain and inflammation that follow surgical and dental procedures in a patient, where the method comprises the step of administering to the patient a therapeutically effective amount of a PPAR-δ agonist. In diseases that are auto-immune in nature, including, but not limited to, rheumatoid arthritis, juvenile rheumatoid arthritis, systemic lupus erythematosus, and many other degenerative joint diseases and connective tissue diseases, administration of a PPAR-δ agonist will, according to the instant invention, diminish the pathological inflammatory responses associated with these diseases. The invention is particularly useful in the treatment of rheumatoid arthritis.

Adjuvant-induced arthritis is a well-established in vivo model of inflammatory diseases or conditions, such as degenerative joint disease, connective tissue diseases, autoimmune diseases, rheumatoid arthritis, juvenile rheumatoid arthritis, and other kinds of inflammation. Adjuvant-induced arthritis is used for the assessment of compounds that are potential candidates for use as anti-inflammatory and/or immunoregulatory compounds. PPAR agonists (α,γ, and δ) and PPAR-γ partial agonists and antagonists have been investigated herein for their utility in the treatment of adjuvant-induced arthritis in rats. Only PPAR-δ agonists showed utility in this animal model, whereas all PPAR-γ ligands and all PPAR-α ligands were ineffective.

The data presented herein demonstrate that PPAR-δ agonists can have utility for treating, preventing, controlling or reducing the risk of contracting one or more diseases or conditions selected from rheumatoid arthritis, juvenile rheumatoid arthritis, systemic lupus erythematosus, osteoarthritis, degenerative joint disease, one or more connective tissue diseases, ankylosing spondylitis, bursitis, Sjogren's syndrome, psoriasis, psoriatic arthritis, neuralgia, synovitis, glomerulonephritis, vasculitis, sacoidosis, inflammations that occur as sequellae to influenza, the common cold and other viral infections, gout, contact dermatitis, low back and neck pain, dysmenorrhea, headache, toothache, sprains, strains, myositis, burns, injuries, and pain and inflammation that follow surgical and dental procedures in a patient, where the method of treatment comprises the steps of first identifying a mammalian patient in need of treatment, including preventive treatment, and then administering a therapeutically effective amount of a PPAR-δ agonist, or a pharmaceutically acceptable salt thereof, to the patient. The patient is a mammal, and preferably is a human.

In preferred embodiments, the PPAR-δ compounds may be used to treat, prevent, control or reduce the risk of contracting one or more auto-immune diseases or disorders selected from systemic lupus erythematosus, rheumatoid arthritis, juvenile rheumatoid arthritis, glomerulonephritis, nephritis, vasculitis, sacoidosis, degenerative joint disease, Sjogren's syndrome, psoriasis, psoriatic arthritis, dysmenorrhea, myositis, neuralgia, synovitis, ankylosing spondylitis and bursitis, by the steps of first identifying a mammalian patient in need of treatment, including preventive treatment, and then administering a therapeutically effective amount of a PPAR-δ agonist, or a pharmaceutically acceptable salt thereof, to the patient.

In a preferred embodiment, the PPAR-δ agonist may be used to treat, prevent, control, or reduce the risk of contracting rheumatoid arthritis, juvenile rheumatoid arthritis, systemic lupus erythematosus, osteoarthritis, degenerative joint disease, one or more connective tissue diseases, ankylosing spondylitis, and bursitis, by the steps of first identifying a mammalian patient in need of treatment, including preventive treatment, and then administering a therapeutically effective amount of a PPAR-δ agonist, or a pharmaceutically acceptable salt thereof, to the patient.

In a preferred embodiment, the PPAR-δ agonist may be used to treat, prevent, control, or reduce the risk of contracting rheumatoid arthritis, juvenile rheumatoid arthritis, systemic lupus erythematosus, osteoarthritis, degenerative joint disease, and one or more connective tissue diseases, by the steps of first identifying a mammalian patient in need of treatment, including preventive treatment, and then administering a therapeutically effective amount of a PPAR-δ agonist, or a pharmaceutically acceptable salt thereof, to the patient.

In particularly preferred embodiments, the PPAR-δ compounds may be used to treat, prevent, control or reduce the risk of contracting rheumatoid arthritis or juvenile rheumatoid arthritis by the steps of first identifying a mammalian patient in need of treatment, including preventive treatment, and then administering a therapeutically effective amount of a PPAR-δ agonist, or a pharmaceutically acceptable salt thereof, to the patient.

In another particularly preferred embodiments, the PPAR-δ compounds may be used to treat, prevent, control or reduce the risk of contracting osteoarthritis or degenerative joint disease by the steps of first identifying a mammalian patient in need of treatment, including preventive treatment, and then administering a therapeutically effective amount of a PPAR-δ agonist, or a pharmaceutically acceptable salt thereof, or a prodrug thereof, to the patient.

PPAR-δ agonists are known in the art. For example, see WO 97/28115, WO 97/28149, WO 97/27857, WO 97/28137, WO 97/27847, WO 98/27974, and WO 01/00603

Dosage and Administration

The PPAR-δ agonists of the invention can be administered in such oral forms as tablets, capsules (each of which includes sustained release or timed release formulations), pills, powders, granules, elixers, tinctures, suspensions, syrups, and emulsions. Likewise, they may be administered in intravenous (bolus or infusion), intraperitoneal, subcutaneous, or intramuscular form, all using forms well known to those of ordinary skill in the pharmaceutical arts.

The PPARδ agonists can be administered in the form of a depot injection or implant preparation which may be formulated in such a manner as to permit a sustained release of the active ingredient. The active ingredient can be compressed into pellets or small cylinders and implanted subcutaneously or intramuscularly as depot injections or implants. Implants may employ inert materials such as biodegradable polymers or synthetic silicones, for example, Silastic, silicone rubber or other polymers manufactured by the Dow-Corning Corporation.

The PPAR-δ agonists can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The PPAR-δ agonists may also be delivered by the use of monoclonal antibodies as individual carriers to which the compound molecules are coupled. The PPAR-δ agonists may also be coupled with soluble polymers as targetable drug carriers. Such polymers can include, but are not limited to, polyvinlypyrrolidinone-pyran copolymer, poly(hydroxypropyl)methacrylamide-phenol copolymer, polyhydroxyethylaspartamide-phenol copolymer, or polyethyleneoxide-polylysine copolymer substituted with palmitoyl residues. Furthermore, the PPAR-δ agonists may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross-linked or amphipathic block copolymers of hydrogels.

The dosage regimen utilizing the PPAR-δ agonists is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, alleviate, coontrol or stop the symptons and the progress of the condition.

Oral administration is the preferred route of drug delivery when oral administration is practicable. Oral dosages of the PPAR-δ agonists, when used for the indicated effects, will generally range between about 0.001 mg per kg of body weight per day (mg/kg/day) to about 100 mg/kg/day, and preferably about 0.01-10 mg/kg/day (unless specified otherwise, amounts of active ingredients are on the basis of a neutral molecule, which may be a free acid or free base). For example, an 80 kg patient would receive between about 0.08 mg/day and 8 g/day, and preferably between about 0.8 mg/day and 800 mg/day. A suitably prepared medicament for once a day administration would thus contain between 0.08 mg and 8 g, and preferably between 0.8 mg and 800 mg. Advantageously, the PPAR-δ agonists may be administered in divided doses of two, three, or four times daily. For administration twice a day, a suitably prepared medicament as described above would contain between 0.04 mg and 4 g, and preferably between 0.4 mg and 400 mg. Dosages outside of the aforementioned ranges may be necessary in some cases. Carrier would generally be added in an amount that would constitute about 5%-95% of the total composition. Examples of daily dosages that may be given in the range of 0.08 mg -8 g per day include 0.1 mg, 0.5 mg, 1 mg, 5 mg, 10 mg, 25 mg, 50 mg, 100 mg, 200 mg, 300 mg, 400 mg, 500 mg, 600 mg, 800 mg, 1 g, 2 g, 4 g and 8 g. These would be divided into smaller doses if administered more than once per day (e.g. one-half the amount in each administration if the drug is taken twice daily).

Intravenously or subcutaneously, the patient would receive the injected doses in a quantity that would deliver the active ingredient in approximately the quantities described above. The quantities may be adjusted to account for differences in the efficiency of delivery that results from the use of a mode of delivery that bypasses the digestive system. Such quantities may be administered in a number of suitable ways, e.g. large volumes of low concentrations of active ingredient during one extended period of time or several times a day, low volumes of high concentrations of active ingredient during a short period of time, e.g. once a day. Typically, a conventional intravenous formulation may be prepared which contains a concentration of active ingredient of between about 0.01-1.0 mg/ml, such as for example 0.1 mg/ml, 0.3 mg/ml, or 0.6 mg/ml, and administered in amounts per day equivalent to the amounts per day stated above. For example, an 80 kg patient, receiving 8 ml twice a day of an intravenous formulation having a concentration of active ingredient of 0.5 mg/ml, receives 8 mg of active ingredient per day. Glucuronic acid, L-lactic acid, acetic acid, citric acid or any pharmaceutically acceptable acid/conjugate base with reasonable buffering capacity in the pH range acceptable for intravenous administration may be used as buffers. Consideration should be given to the solubility and chemical compatibility of the drug in choosing an appropriate excipient. Subcutaneous formulations, preferably prepared according to procedures well known in the art at a pH in the range between 7.0 and 7.4, also include suitable buffers and isotonicity agents. They are formulated to deliver a daily dose of PPAR-δ agonist in one or more daily subcutaneous administrations, e.g., one, two or three times each day. The choice of appropriate buffer and pH of a formulation, depending on solubility of the drug to be administered, is readily made by a person having ordinary skill in the art.

The compounds can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

The PPAR-δ agonists are typically administered as active ingredients in admixture with suitable pharmaceutical diluents, excipients or carriers (collectively referred to herein as “carrier” materials) suitably selected with respect to the intended form of administration, that is, oral tablets, capsules, elixers, syrups and the like, and consistent with convention pharmaceutical practices.

For instance, for oral administration in the form of a tablet or hard capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. For oral administration in liquid form, the oral drug components can be combined with any oral, non-toxic, pharmaceutically acceptable inert carrier such as ethanol, glycerol, water and the like. The oral dosage form may also be suspended in an oil, such as a vegetable oil, which could be, without limitation, arachis oil, olive oil, sesame oil or coconut oil, or a mineral oil, such as paraffin oil. Moreover, when desired or necessary, suitable binders, lubricants, disintegrating agents and coloring agents can also be incorporated into the mixture. Suitable binders include starch, gelatin, natural sugars such as glucose or beta-lactose, corn-sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

Definitions

“Pharmaceutically acceptable salts” means non-toxic salts of the compounds employed in this invention which are generally prepared by reacting the free acid with a suitable organic or inorganic base. Examples of salt forms of PPAR-δ agonists may include, but are not limited to, acetate, benzenesulfonate, benzoate, bicarbonate, bisulfate, bitartrate, borate, bromide, calcium, calcium edetate, camsylate, carbonate, chloride, clavulanate, citrate, dihydrochloride, edetate, edisylate, estolate, esylate, fumarate, gluceptate, gluconate, glutamate, hexylresorcinate, hydrabamine, hydrobromide, hydrochloride, hydroxynapthoate, isothionate, lactate, lactobionate, laurate, malate, maleate, mandelate, mesylate, methylsulfate, mucate, oleate, oxalate, pamaote, palmitate, panthothenate, phosphate/diphosphate, polygalacturonate, potassium, salicylate, sodium, stearate, subacetate, succinate, tannate, tartrate, teoclate, tosylate, and valerate. Examples of salt forms of COX-2 inhibitors include but are not limited to salts derived from inorganic bases including aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanol amine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Unless defined otherwise, “therapeutically effective amount” means that amount of a drug or pharmaceutical agent that will elicit the biological or medical response of a tissue, a system, animal or human that is being sought by a researcher, veterinarian, medical doctor or other clinician.

Unless defined otherwise, “prophylactically effective amount” means that amount of a pharmaceutical drug that will prevent or reduce the risk of occurrence of the biological or medical event that is sought to be prevented in a tissue, a system, animal or human by a researcher, veterinarian, medical doctor or other clinician.

Combination Therapy

Similarly, PPAR-δ agonists may be useful as a partial or complete substitute for conventional NSAID's in preparations where NSAID's are presently co-administered with other agents or ingredients. Thus in further aspects, the invention encompasses pharmaceutical compositions for treating inflammatory diseases as defined above comprising a non-toxic therapeutically effective amount of a PPAR-6 agonist as defined above and one or more ingredients such as another pain reliever; an NSAID; a potentiator including caffeine; an H2-antagonist; aluminum or magnesium hydroxide; simethicone; a decongestant; an antitussive; a diuretic; and a sedating or non-sedating antihistamine. In addition the invention encompasses a method of treating inflammatory diseases comprising administration to a patient in need of such treatment a non-toxic therapeutically effect amount of a PPAR-δ agonist, optionally co-administered with one or more of such ingredients as listed immediately above.

Examples of antitussives include codeine, hydrocodone, caramiphen, carbetapentane, and dextramethorphan.

Examples of decongestants include phenylephrine, phenylpropanolamine, pseudophedrine, oxymetazoline, ephinephrine, naphazoline, xylometazoline, propylhexedrine, or levo-desoxyephedrine.

Examples of pain relievers include acetominophen and phenacetin.

Combination of PPAR-δ Agonist And NSAID

The instant invention also involves a novel combination therapy comprising the administration of a therapeutically effective amount of an NSAID such as a selective or non-selective COX-2 inhibitor in combination with a therapeutically effective amount of a PPAR-δ agonist to a mammal, and more particularly, to a human. The combination therapy is used to treat inflammation and inflammatory diseases.

The pharmaceutical combinations comprising a PPAR-δ agonist in combination with an NSAID such as a COX-2 inhibitor include single pharmaceutical dosage formulations which contain both the PPAR-δ agonist and the NSAID in a single dose, as well as formulations in which each active agent is administered in its own separate pharmaceutical dosage formulation. Where separate dosage formulations are used, the PPAR-δ agonist and the NSAID can be administered at essentially the same time, i.e., concurrently, or at separately staggered times, i.e, sequentially. The “instant pharmaceutical combination” is understood to include all these regimens. Administration in these various ways are suitable for the present invention as long as the beneficial pharmaceutical effect of the PPAR-δ agonist and the NSAID are realized by the patient at substantially the same time. Such beneficial effect is preferably achieved when the target blood level concentrations of each active drug are maintained at substantially the same time. It is preferred that the PPAR-δ agonist and the NSAID be co-administered concurrently on a once-a-day dosing schedule; however, varying dosing schedules, such as the PPAR-δ agonist once per day and the NSAID once, twice or more than twice per day, or the NSAID once per day and the PPAR-δ agonist once, twice or more than twice per day, is also encompassed herein. A single oral dosage formulation comprised of both the PPAR-δ agonist and the NSAID is preferred. A single dosage formulation will provide convenience for the patient.

The instant invention also provides pharmaceutical compositions comprised of a therapeutically effective amount of an NSAID, or a pharmaceutically acceptable salt thereof, in combination with a therapeutically effective amount of a PPAR-δ agonist, or a pharmaceutically acceptable salt thereof, and a pharmaceutically acceptable carrier. One embodiment of the instant compositions is a single composition adapted for oral administration comprised of a therapeutically effective amount of a COX-2 inhibitor in combination with a therapeutically effective amount of a PPAR-δ agonist and a pharmaceutically acceptable carrier. The combination can also be administered in separate dosage forms, each having one of the active agents. If administered in separate dosage forms, the separate dosage forms are administered such that the beneficial effect of each active agent is realized by the patient at substantially the same time.

NSAIDs

Common NSAIDs include salicylates such as aspirin, sodium salicylate, choline salicylate, salicylsalicylic acid, diflunisal, and salsalate; indoleacetic acids such as indomethacin and sulindac; pyrazoles such as phenylbutazone, oxyphenbutazone; pyrrolealkanoic acids such as tolmetin; phenylacetic acids such as ibuprofen, feroprofen, flurbiprofen, and ketoprofen; fenamates such as mefanamic acid, and meclofenamate; oxicams such as piroxicam; and naphthaleneacetic acids such as naproxen. These are all non-selective NSAID's, inhibiting both cyclooxygenase-1 (COX-1) and cyclooxygenase-2 (COX-2) receptors.

COX-2 Inhibitors

Selective cyclooxygenase-2 (COX-2) inhibitors are particularly advantageous for patients who are sensitive to the side effects of cyclooxygenase-1 (COX-1) inhibition that results when non-selective NSAID's are administered. Examples of COX-2 inhibitore that may be advantageously used in combination therapy with PPAR-δ agonists include rofecoxib, etoricoxib, celecoxib, parecoxib and valdecoxib.

The dosage regimen utilizing a PPAR-δ agonist in combination with the NSAID is selected in accordance with a variety of factors including type, species, age, weight, sex and medical condition of the patient; the severity of the condition to be treated; the route of administration; the renal and hepatic function of the patient; and the particular compound or salt or ester thereof employed. Since two different active agents are being used together in a combination therapy, the potency of each of the agents and the interactive effects achieved by combining them together must also be taken into account. A consideration of these factors is well within the purview of the ordinarily skilled clinician for the purpose of determining the therapeutically effective or prophylactically effective dosage amounts needed to prevent, counter, or arrest the progress of the condition.

Administration of the drug combination to the patient includes both self-administration and administration to the patient by another person.

Additional active agents may be used in combination with the NSAID and PPAR-δ agonist in a single dosage formulation, or may be administered to the patient in a separate dosage formulation, which allows for concurrent or sequential administration. Examples of additional active agents which may be employed include HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors; probucol; niacin; PPAR-α agonists, including fibrates such as clofibrate, fenofibrate, and gemfibrizol; PPAR-γ agonists and PPAR-α/γ dual agonists, which are insulin sensitizers; cholesterol absorption inhibitors; bile acid sequestrants; LDL (low density lipoprotein) receptor inducers; vitamin B₆ (also known as pyridoxine) and the pharmaceutically acceptable salts thereof such as the HCl salt; vitamin B₁₂ (also known as cyanocobalamin); β-adrenergic receptor blockers; folic acid or a pharmaceutically acceptable salt or ester thereof such as the sodium salt and the methylglucamine salt; and anti-oxidant vitamins such as vitamin C and E and beta carotene.

An additional embodiment of the instant invention involves a kit comprised of an NSAID such as a COX-2 inhibitor in an oral dosage formulation and a PPAR-δ agonist in a separate oral dosage formulation.

One example of this embodiment is a kit comprised of an oral dosage formulation of a COX-2 inhibitor and an oral dosage formulation of a PPAR-δ agonist. The packaging for the kit could be designed and manufactured in a variety of ways. A preferred example is a blister package containing rows of a COX-2 inhibitor tablet and a PPAR-δ agonist tablet placed side by side on the same blister card, each of the two tablets in its own blister bubble, with calendar or similar type markings on the card that convey to the user that one “pair” of tablets (i.e., one COX-2 inhibitor tablet and one PPAR-δ agonist tablet) is to be ingested per day.

### EXAMPLES

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

### Assays

As an approach to identifying human PPAR agonists, a Scintillation Proximity Assay was used to characterize PPAR-γ, PPAR-δ, and PPAR-α ligands. In these assays, the ability of test compounds to displace binding of an MRL radioligand, [³H₂](3-(4-(3-phenyl-7-propyl-6-benz-[4,5]-isoxazoloxy)butyloxy))phenylacetic acid (compound A), from recombinant human PPAR-(X or recombinant human PPAR-γ was measured. In order to characterize the relative ability of test compounds to bind to recombinant human PPAR-δ, the displacement of radiolabeled [³H_{2]3}-chloro-4-(3-(7-propyl-3-trifluoromethyl-6-benz-[4,5]-isoxazoloxy)propylthio)phenylacetic acid (compound B) was employed. Compounds A and B (non-labelled) are disclosed in WO 97/28137 as Examples 62 and 20, respectively. Radiolabeled Compounds A and B are made by modification of the methods disclosed in WO 97/28137 or labelling of the compounds.

### Binding Assays

A. Preparation of Recombinant Human PPARγ, PPARδ, and PPARα

Human PPARγ₂, human PPARδ and human PPARα were expressed as GST-fusion proteins in E. coli. The full length human cDNA for PPARγ ₂ was subcloned into the pGEX-2 T expression vector (Amersham Pharmacia Biotech, Inc.). The full length human cDNAs for PPARδ and PPARα were subcloned into the pGEX-KT expression vector (Amersham Pharmacia Biotech, Inc.). E. coli containing the respective plasmids were propagated, induced, and harvested by centrifugation. The resuspended pellet was broken in a French press and debris removed by centrifugation. Recombinant human PPAR receptors were purified by affinity chromatography on glutathione sepharose. After application to the column, and one wash, receptor was eluted with glutathione. Glycerol (10%) was added to stabilize the receptor and aliquots were stored at −80° C.

B. [³H₂]-Compound A PPARSPA (PPARγ)

For each assay, an aliquot of human GST-PPARγ receptor was incubated in a final volume of 100 μl SPA buffer (10 mM Tris, pH 7.2, 1 mM EDTA, 10% glycerol, 10 mM Na molybdate, 1 mM dithiothreitol and 2 μg/ml benzamidine) containing 1.25 mg/ml yttrium silicate protein-A coated SPA beads (Amersham Pharmacia Biotech, Inc.), 8.3 μg/ml anti-GST antibody (Amersham Pharmacia Biotech, Inc.) 0.1% non-fat dry milk and 5 nM [³H₂]Compound A (34.3 Ci/mmole), ±test compound. After incubation for ˜16 h at 15° C. with shaking, the assay plates were counted in a Packard Topcount with quench correction. In this assay the K_{D} of Compound A for PPARγ is ≈2.7 nM.

C. [³H₂]Compound A PPARSPA Assay (PPARα)

For each assay, an aliquot of human GST-PPARα receptor was incubated in a final volume of 100 μl SPA buffer (10 mM Tris, pH 7.2, 1 mM EDTA, 10% glycerol, 10 mM Na molybdate, 1 mM dithiothreitol and 2 μg/ml benzamidine) containing 1.25 mg/ml yttrium silicate protein-A coated SPA beads (Amersham Pharmacia Biotech, Inc.), 8.3 μg/ml anti-GST antibody (Amersham Pharmacia Biotech, Inc.) 0.1% non-fat dry milk and 5 nM [³H₂]Compound A (34.3 Ci/mmole), ±test compound. After incubation for ˜16 h at 15° C. with shaking, the assay plates were counted in a Packard Topcount with quench correction. In this assay the K_{D} of Compound A for PPARα is ≈10 nM.

D. [³H₂]Compound B PPARSPA Assay (PPARδ):

For each assay, an aliquot of human GST-PPARδ receptor was incubated in a final volume of 100 μl SPA buffer (10 mM Tris, pH 7.2, 1 mM EDTA, 10% glycerol, 10 mM Na molybdate, 1 mM dithiothreitol, 2 μg/ml benzamidine and 0.5 mM PMSF) containing 1.25 mg/ml yttrium silicate protein-A coated SPA beads (Amersham Pharmacia Biotech, Inc.), 8.3 μg/ml anti-GST antibody (Amersham Pharmacia Biotech, Inc.) 0.1% non-fat dry milk and 2.5 nM [³H₂]Compound B (13.4 Ci/mmole), ±test compound. After incubation for ˜16 h at 15° C. with shaking, the assay plates were counted in a Packard Topcount with quench correction. In this assay the K_{D} of Compound B for PPARδ is ≈1 nM.

### Gal-4 hPPAR Transactivation Assays

The chimeric receptor expression constructs, pcDNA3-hPPARγ/GAL4, pcDNA3-hPPARδ/GAL4, pcDNA3-hPPARα/GAL4 were prepared by inserting the yeast GAL4 transcription factor DBD adjacent to the ligand binding domains (LBDs) of hPPARγ, hPPARδ, hPPARα, respectively. The reporter construct, pUAS(5×)-tk-luc was generated by inserting 5 copies of the GAL4 response element upstream of the herpes virus minimal thymidine kinase promoter and the luciferase reporter gene. pCMV-lacZ contains the galactosidase Z gene under the regulation of the cytomegalovirus promoter. COS-1 cells were seeded at 12×10³ cells/well in 96 well cell culture plates in high glucose Dulbecco's modified Eagle medium (DMEM) containing 10% charcoal stripped fetal calf serum (Gemini Bio-Products, Calabasas, Calif.), nonessential amino acids, 100 units/ml Penicillin G and 100 mg/ml Streptomycin sulfate at 37° C. in a humidified atmosphere of 10% CO₂. After 24 h, transfections were performed with Lipofectamine (GIBCO BRL, Gaithersburg, Md.) according to the instructions of the manufacturer. Briefly, transfection mixes for each well contained 0.48 μl of Lipofectamine, 0.00075 μg of pcDNA3-PPAR/GAL4 expression vector, 0.045 μg of pUAS(5×)-tk-luc reporter vector and 0.0002 μg of pCMV-lacZ as an internal control for transactivation efficiency. Cells were incubated in the transfection mixture for 5 h at 37° C. in an atmosphere of 10% CO₂. The cells were then incubated for ˜48 h in fresh high glucose DMEM containing 5% charcoal stripped fetal calf serum, nonessential amino acids, 100 units/ml Penicillin G and 100 mg/ml Streptomycin sulfate ±increasing concentrations of test compound. Since the compounds were solubilized in DMSO, control cells were incubated with equivalent concentrations of DMSO; final DMSO concentrations were ≦0.1%, a concentration which was shown not to effect transactivation activity. Cell lysates were produced using Reporter Lysis Buffer (Promega, Madison, Wis.) according to the manufacturer's instructions. Luciferase activity in cell extracts was determined using Luciferase Assay Buffer (Promega, Madison, Wis.) in an ML3000 luminometer (Dynatech Laboratories, Chantilly, Va.). β-galactosidase activity was determined using β-D-galactopyranoside (Calbiochem, San Diego, Calif.).

### EXAMPLE

The following table (Table A) shows the structures of the PPAR-α, PPAR-γ and PPAR-δ ligands that were tested for activity for preventing, controlling or reducing inflammation resulting from adjuvant-induced arthritis in female Lewis rats. The kind of PPAR activity of each compound is also shown in Table A. Table B shows the IC₅₀ and EC₅₀ data that were used to classify the ligands according to their PPAR activity (e.g. PPAR-α agonist, PPAR-δ agonist, and the like). The anti-inflammatory NSAID indomethacin was also tested on rats in which adjuvant-induced arthritis was induced as a control.

Compound 1, generally known as GW 501516, is published in WO 01/00603, Compound 2 is {2-[2-(4-phenoxy-2-propylphenoxy)ethyl]-1H-indol-5-yl}acetic acid and is Example 4 in WO 98/27974. Compound 3 is described in U.S. Pat. No. 5,081,138. Compounds 1-3 can all be made by methods disclosed in the three references. Compounds 4, 5, and 6 and indomethacin are all well known in the art and can be obtained commercially or made by well known methods.

TABLE A

| Compound Number | Structure | PPAR Activity |
|---|---|---|
| Compound 1 GW501516 | 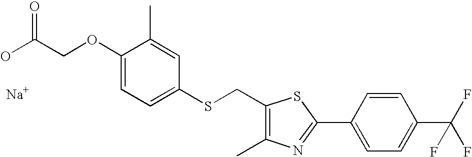 | Delta Agonist |
| Compound 2 | 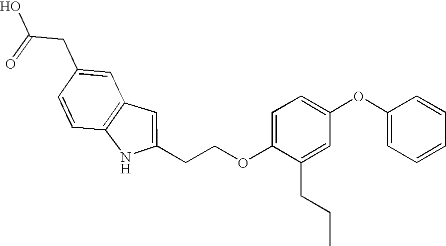 | Gamma Agonist |
| Compound 3 | 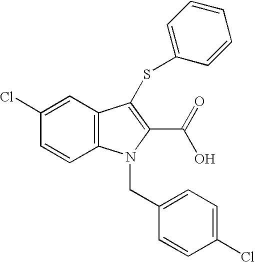 | Gamma Antagonist |
| Compound 4 WY-14643 | 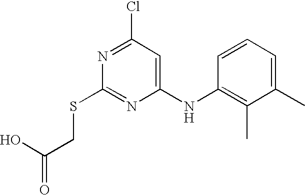 | Alpha Agonist |
| Compound 5 Fenofibrate | 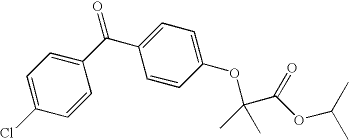 | Alpha Agonist |
| Compound 6 Rosiglitazone | 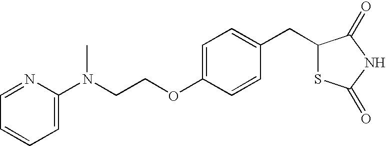 | Gamma Agonist |

TABLE B

| | IC50 (μM: SPA or BND) | | | EC50 (μM: transactivation) | | |
|---|---|---|---|---|---|---|
| Compound | Alpha | Delta | Gamma | Alpha | Delta | Gamma |
| Compound 1 | 8.52 | <0.001 | 11.6 | 0.52/1.26 | 0.03 | >3/>3 |
| Compound 2 | 1.48 | 15 | 0.097 | 1.05/1.2 | 3/3 | 0.18/0.05 |
| Compound 3 | >10 | >50 | 0.011 | | | 0.05/0.15 |
| Compound 4 | 29.2 | >150 | 50.7 | 0.16/0.3 | >30 | >50 |
| Compound 5 | | | | | | |
| Compound 6 | >50 | >50 | 0.207 | >3 | >50 | 0.01 |

Development and Severity of Adjuvant-Induced Arthritis in Female Lewis Rats

One hundred, 7 week old, female Lewis rats (Charles River-Portage) (body weight 139-172 g) were weighed, ear marked, and assigned to groups of 10 rats each such that the body weights were equivalent within each group; a negative control group in which arthritis was not induced, a vehicle (0.5% methocel in sterile distilled water) control group, a positive control group administered indomethacin per os (p.o.) at a daily dose of 2 mg/kg q.d, Compound 2 at 30 mg/kg p.o. q.d, Compound 3 at 50 mg/kg p.o. q.d, Compound 4 at 30 mg/kg p.o. q.d, Compound 5 at 106 mg/kg p.o. q.d, Compound 6 at 30 mg/kg p.o. q.d and Compound 1 at 30 mg/kg p.o. q.d. Nine groups of 10 rats each were injected with an emulsion containing 0.5 mg of Mycobacterium butyricum in 0.1 ml of light mineral oil, and a negative control group of 10 rats was not injected with adjuvant. Body weights, primary (injected) and contralateral (non-injected) paw volumes (determined by mercury displacement plethysmography) and lateral radiographs (obtained under Ketamine and Xylazine anesthesia) were determined before (day-1) and at various time points including 21 days following adjuvant injection. The rats were anesthetized with an intramuscular injection of 0.03-0.1 ml of a combination of Ketamine (87 mg/kg) and Xylazine (13 mg/kg) for radiographs and injection of adjuvant. Lateral to medial radiographs were made of both hind paws on days 0 and 21 for using the Faxitron (45 kVp, 30 seconds or 45 kVp, 1 second) and Kodak X-OMAT TL film, and were developed in an automatic processor. Radiographs were evaluated for changes in the soft and hard tissues by a person who had experience in reading these kinds of radiographs and was blinded to experimental treatment. The following radiographic changes were graded numerically according to severity: increased soft tissue volume (0-4), narrowing or widening of joint spaces (0-5), subchondral erosion (0-3), periosteal reaction (0-4), osteolysis (0-4), subluxation (0-3), and degenerative joint changes (0-3). Specific criteria were used to establish the numerical grade of severity for each radiographic change. The maximum possible score per foot was 26. Compounds or vehicle were administered prophylactically beginning post injection of adjuvant and continuing for 21 days. The Compounds were prepared weekly, refrigerated in the dark until used, and vortex mixed immediately prior to administration. Following euthanasia by carbon dioxide inhalation blood was obtained by cardiac puncture for determination of plasma trough (rats 1-5) and 2 hrs post dose (rats 6-10) levels from rats administered Compound 1, and vehicle. The liver, thymus and spleens of all rats were removed and weighed and both hind paws were removed and radiographed.

Two-factor (‘treatment’ and ‘time’) analysis of variance with repeated measures on ‘time’ were applied to the percent changes for body weight and foot volumes and to the rank-transformed radiographic total scores. A post hoc Dunnett's test was conducted to compare the effect of treatments to vehicle. A one-way analysis of variance was applied to the thymic and spleen weights followed by the Dunnett's test to compare the effect of treatments to vehicle.

Results:

One rat from the control group died from anesthesia on day 0. Thus, the control group contained 9 rats. Rats administered the PPAR γ-agonist Compound 2, the PPAR γ-antagonist Compound 3, the PPAR α-agonist Compound 4, the PPAR α-agonist Compound 5, and the PPAR γ-agonist Compound 6 were euthanized on day 14 because the compounds failed to prevent swelling of either the primary or secondary paws.

Body weight

Compounds or vehicle (0.5% Methocel in sterile water) were administered by oral gavage once daily for 21 days following induction of arthritis in female Lewis rats via the sub plantar injection of 0.1 ml of light mineral oil containing 0.5 mg of Mycobacterium butyricum. Control animals were not injected with adjuvant. Body weights were measured 1 day prior to (i.e. day 0) and at 14 and 21 days post induction of arthritis. The data are presented in Table 1. The data in Table 1 represent the mean increase or decrease in body weight ±1 standard deviation for groups of 9 to 10 animals.

Control rats (animals not injected with adjuvant) gained weight during the course of the study and differed significantly from all rats with adjuvant-induced arthritis. As a manifestation of the systemic nature of the model, rats with adjuvant-induced arthritis lost weight. Rats treated prophylactically with Compound 1 (30 mg/kg q.d.) or indomethacin gained more weight than did vehicle-treated animals in this study (Table 1).

Paw Swelling:

Compounds or vehicle (0.5% Methocel in sterile water) were administered by oral gavage once daily for 21 days following induction of arthritis in female Lewis rats via the sub plantar injection of 0.1 ml of light mineral oil containing 0.5 mg of Mycobacterium butyricum. Control animals were not injected with adjuvant.

Injected foot volumes were measured by mercury displacement plethysmography prior to induction of arthritis, and on days 4, 14 and 21 post induction of arthritis. The data represent the mean increase in foot volume ±1 standard deviation for groups of 9 to 10 animals. The extent of paw swelling in compound-treated animals was compared to the mean of that in the vehicle-treated group, and percentage inhibition was calculated for each dose level of compound. When the extent of paw swelling for an individual animal was greater than the mean extent of swelling for the vehicle control group, the percentage inhibition was recorded as zero.

Contralateral foot volumes were measured by mercury displacement plethysmography prior to, and at days 14 and 21 post induction of arthritis. The data represent the mean increase in foot volume ±1 standard deviation for groups of 9 to 10 animals. The extent of paw swelling in compound-treated animals was compared to the mean of that in the vehicle-treated group, and percentage inhibition was calculated for each dose level of compound. When the extent of paw swelling for an individual animal was greater than the mean extent of swelling for the vehicle control group the percentage inhibition was recorded as zero.

Prophylactic administration of Compound 1 (30 mg/kg q.d.) inhibited the day 21 swelling of the contralateral (secondary) paw but did not affect the swelling of the primary (injected) paw. Swelling of the secondary paw of rats administered Compound 1 was significantly less than that in vehicle-treated rats. The inhibition of paw swelling by Compound 1 was similar to that seen with indomethacin (2 mg/kg q.d.). Paw volume data are summarized in Tables 2 (primary) and 3 (secondary).

Spleen, Thymus and Liver Weight:

Organ weights were obtained at necropsy 21 days post induction of arthritis. Data represent the mean weights of organs in mg ±1 standard deviation for groups of 9 to 10 animals.

Thymic involution and splenomegaly are consistent findings in AIA. None of the compounds protected rats with adjuvant arthritis against the development of splenomegaly. The degree of thymic involution was less severe in animals treated with Compound 1 or indomethacin than animals treated with vehicle. The Livers of animals administered Compound 1 were significantly heavier than those of rats treated with vehicle. Organ weight data are summarized in table 4.

Radiographic Scores:

Total radiographic scores were derived from examination of lateral radiographs obtained prior to, and on day 21 post induction of arthritis. Data represent the mean scores ±1 standard deviation for groups of 9 to 10 animals.

The radiographic total scores for both hind paws of vehicle treated rats with AIA were significantly greater than those of non-adjuvant control rats on day 21. The radiographic total scores for the contralateral paws of rats administered Compound 1 or indomethacin and the primary paws of rats administered indomethacin on day 21 were significantly less than those of vehicle-treated rats. Radiographic data are summarized in table 5.

TABLE 1
The effects of various treatments on change in body weight
in grams of rats with Adjuvant-Induced Arthritis

| Treatment | Day 14-0 | Day 21-0 |
|---|---|---|
| Control | 38.0 ± 5.6⁺⁺ | 50.6 ± 6.3⁺⁺ |
| Vehicle (0.5% Methocel- | −13.9 ± 4.6**⁺⁺ | −13.2 ± 6.2** |
| p.o. q.d.) | | |
| Compound 2 (p.o. q.d.) | 0.8 ± 7.3**⁺⁺ | |
| 30 mg/kg/day | | |
| Compound 3 (p.o. q.d.) | −6.6 ± 6.1** | |
| 50 mg/kg/day | | |
| Compound 4 (p.o. q.d.) | −15.2 ± 6.8** | |
| 30 mg/kg/day | | |
| Compound 5 (p.o. q.d.) | −7.6 ± 5.3** | |
| 100 mg/kg/day | | |
| Compound 6 (p.o. q.d.) | 0.4 ± 6.4**⁺⁺ | |
| 30 mg/kg/day | | |
| Compound 1 (p.o. q.d.) | 1.9 ± 6.6**⁺⁺ | 2.9 ± 7.4**⁺⁺ |
| 30 mg/kg/day | | |
| Indomethacin (p.o. q.d.) | 8.6 ± 7.7**⁺⁺ | 11.6 ± 8.0**⁺⁺ |
| 2 mg/kg/day | | |

(*, **)significant difference from Control with p at 0.05 (*) or 0.01(**) with Dunnett's test
(+, ++)significant difference from Vehicle with p at 0.05 (⁺) or 0.01(⁺⁺) with Dunnett's test

TABLE 2
Change in Primary Foot Volumes on days 4, 14 & 21 and % Inhibition
Compared to Vehicle

| | Day 4-0 | | Day 14-0 | | Day 21-0 | |
|---|---|---|---|---|---|---|
| Treatment | μL | % I | μL | % I | μL | % I |
| Control | | | 149 ± 81⁺⁺ | | 205 ± 96⁺⁺ | |
| Vehicle (0.5% Methocel- | 1954 ± 271 | | 2326 ± 492** | | 2395 ± 657** | |
| po qd) | | | | | | |
| Compound 2 (p.o. q.d.) | 1642 ± 386 | 18.6% | 1875 ± 418** | 21.3% | | |
| 30 mg/kg/day | | | | | | |
| Compound 3 (p.o. q.d.) | 1853 ± 262 | 0% | 2606 ± 587** | | | |
| 50 mg/kg/day | | | | | | |
| Compound 4 (p.o. q.d.) | 2133 ± 266 | 0% | 2585 ± 733** | | | |
| 30 mg/kg/day | | | | | | |
| Compound 5 (p.o. q.d.) | 1421 ± 212⁺ | 27.3% | 1945 ± 415** | 17.4% | | |
| 100 mg/kg/day | | | | | | |
| Compound 6 (p.o. q.d.) | 1322 ± 404 | 32.3% | 1892 ± 422** | 18.9% | | |
| 30 mg/kg/day | | | | | | |
| Compound 1(p.o. q.d.) | 1510 ± 523⁺ | 23.1% | 1530 ± 659**⁺⁺ | 30.0% | 2612 ± 909** | 12.6% |
| 30 mg/kg/day | | | | | | |
| Indomethacin (p.o. q.d.) | 798 ± 164⁺⁺ | 59.2% | 799 ± 159*⁺⁺ | 65.7% | 499 ± 135⁺⁺ | 79.2% |
| 2 mg/kg/day | | | | | | |

(*, **) significant difference from Control with p at 0.05 (*) or 0.01(**) with Dunnett's test
(+, ++) significant difference from Vehicle with p at 0.05 (⁺) or 0.01(⁺⁺) with Dunnett's test

TABLE 3
Change in Secondary Foot Volumes and % Inhibition Compared
to Vehicle for rats with Adjuvant-Induced Arthritis

| | Day 14-0 | | Day 21-0 | |
|---|---|---|---|---|
| Treatment | μL | % I | μL | % I |
| Control | 177 ± 64⁺⁺ | | 219.0 ± | |
| | | | 78.0⁺⁺ | |
| Vehicle (0.5% Methocel- | 736 ± 283** | | 966.0 ± | |
| po qd) | | | 279.0** | |
| Compound 2 (p.o. q.d.) | 786 ± 220** | 0% | | |
| 30 mg/kg/day | | | | |
| Compound 3 (p.o. q.d.) | 869 ± 291** | 0% | | |
| 50 mg/kg/day | | | | |
| Compound 4 (p.o. q.d.) | 704 ± 421** | 22.1% | | |
| 30 mg/kg/day | | | | |
| Compound 5 (p.o. q.d.) | 824 ± 186** | 0% | | |
| 100 mg/kg/day | | | | |
| Compound 6 (p.o. q.d.) | 1071 ± 364** | 0% | | |
| 30 mg/kg/day | | | | |
| Compound 1 (p.o. q.d.) | 38 ± 161⁺⁺ | 92.4% | 246.0 ± | 77.8% |
| 30 mg/kg/day | | | 498⁺⁺ | |
| Indomethacin (p.o. q.d.) | 275 ± 138⁺⁺ | 62.0% | 201.0 ± | 79.2% |
| 2 mg/kg/day | | | 73.0⁺⁺ | |

significant difference from Control with p at 0.05(*) or 0.01(**) with Dunnett's test
significant difference from Vehicle with p at 0.05(⁺) or 0.01(⁺⁺) with Dunnett's test

TABLE 4
The effects of various treatments on thymus, spleen and liver weights in
milligrams of rats with adjuvant-induced arthritis.

| | Day of | | | |
|---|---|---|---|---|
| Treatment | euthanasia | Thymus (mg) | Spleen (mg) | Liver (mg) |
| Control | 21 | 465.6 ± 84.0⁺⁺ | 517.8 ± 91.1⁺⁺ | 849.0 ± 78.0 |
| Vehicle (0.5% Methocel- | 21 | 172.4 ± 47.8** | 686.0 ± 75.5** | 818.0 ± 63.0 |
| po qd) | | | | |
| Compound 2 (p.o. q.d.) | 15 | 168.7 ± 44.6 | 590.8 ± 82.1 | |
| 30 mg/kg/day | | | | |
| Compound 3 (p.o. q.d.) | 15 | 152.7 ± 27.9 | 720.5 ± 155.1 | |
| 50 mg/kg/day | | | | |
| Compound 4 (p.o. q.d.) | 15 | 146.8 ± 33.4 | 665.1 ± 69.8 | |
| 30 mg/kg/day | | | | |
| Compound 5 (p.o. q.d.) | 15 | 155.3 ± 27.8 | 644.4 ± 159.2 | |
| 100 mg/kg/day | | | | |
| Compound 6 (p.o. q.d.) | 15 | 187.8 ± 50.4 | 634.5 ± 91.4 | |
| 30 mg/kg/day | | | | |
| Compound 1 (p.o. q.d.) | 21 | 275.0 ± 50.4**⁺⁺ | 601.0 ± 52.8 | 1209.0 ± 129.0**⁺⁺ |
| 30 mg/kg/day | | | | |
| Indomethacin (p.o. q.d.) | 21 | 426.0 ± 69.0⁺⁺ | 738.0 ± 124.0** | 866.0 ± 86.0 |
| 2 mg/kg/day | | | | |

(*, **)significant difference from Control with p at 0.05(*) or 0.01(**) with Dunnett's test
(+, ++)significant difference from Vehicle with p at 0.05(⁺) or 0.01(⁺⁺) with Dunnett's test

TABLE 5
The effects of various treatments on radiographic total scores
on day 21 of rats with adjuvant-induced arthritis.

| | Primary | Secondary |
|---|---|---|
| Treatment | (injected) paw | (contralateral) Paw |
| Control | 0.0 ± 0.0⁺⁺ | 0.0 ± 0.0⁺⁺ |
| Vehicle (0.5% Methocel-po qd) | 16.6 ± 3.1** | 10.7 ± 2.8** |
| Compound 1 (p.o. q.d.) | 13.0 ± 7.2** | 1.8 ± 3.3⁺⁺ |
| 30 mg/kg/day | | |
| Indomethacin (p.o. q.d.) | 4.3 ± 1.3⁺⁺ | 3.3 ± 1.3**⁺⁺ |
| 2 mg/kg/day | | |

significant difference from Control with p at 0.05(*) or 0.01(**) with Dunnett's test
significant difference from Vehicle with p at 0.05(⁺) or 0.01(⁺⁺) with Dunnett's test

1. A method for treating, controlling, preventing or reducing the risk of contracting an inflammatory disease or condition in a mammalian patient which comprises the steps of administering to a patient in need of treatment a therapeutically effective amount of a PPAR-δ agonist.

2. The method of claim 1, wherein the inflammatory disease or condition is selected from the group consisting of rheumatoid arthritis, juvenile rheumatoid arthritis, systemic lupus erythematosus, osteoarthritis, degenerative joint disease, one or more connective tissue diseases, ankylosing spondylitis, bursitis, Sjogren's syndrome, psoriasis, psoriatic arthritis, neuralgia, synovitis, glomerulonephritis, vasculitis, sacoidosis, inflammations that occur as sequellae to influenza, the common cold and other viral infections, gout, contact dermatitis, low back and neck pain, dysmenorrhea, headache, toothache, sprains, strains, myositis, burns, injuries, and pain and inflammation that follow surgical and dental procedures in a patient.

3. The method of claim 1, wherein the inflammatory disease or condition is selected from the group consisting of rheumatoid arthritis, juvenile rheumatoid arthritis, systemic lupus erythematosus, osteoarthritis, degenerative joint disease, one or more connective tissue diseases, ankylosing spondylitis, and bursitis.

4. The method of claim 1, wherein the inflammatory disease or condition is selected from the group consisting of rheumatoid arthritis, juvenile rheumatoid arthritis, systemic lupus erythematosus, osteoarthritis, degenerative joint disease, and one or more connective tissue diseases.

5. The method of claim 1, wherein the inflammatory disease or condition is osteoarthritis.

6. The method of claim 1, wherein the inflammatory disease or condition is rheumatoid arthritis.

7. A method for treating, controlling, preventing or reducing the risk of contracting an inflammatory disease or condition in a mammalian patient, which comprises the steps of administering to a patient in need of treatment a therapeutically effective amount of a PPAR-δ agonist and one or more additional therapeutic compounds, wherein said additional therapeutic compounds are selected from the group consisting of a second pain reliever; an NSAID; a potentiator comprising caffeine; an H2-antagonist; aluminum or magnesium hydroxide; simethicone; a decongestant; an antitussive; a diuretic; and a sedating or non-sedating antihistamine.

8. The method of claim 7, wherein said additional therapeutic compounds are selected from the group consisting of a second pain reliever, said second pain reliever being selected from the group consisting of acetominophen and phenacetin; an NSAID that may be a non-selective or selective COX-2 inhibitor; a potentiator comprising caffeine; and H2-antagonist; aluminum or magnesium hydroxide; simethicone; a decongestant comprising one or more ingredients selected from phenylephrine, phenylpropanolamine, pseudophedrine, oxymetazoline, epinephrine, naphazoline, xylometazoline, propylhexedrine, and levo-desoxyephedrine; an antitussive comprising one or more compounds selected from codeine, hydrocodone, caramiphen, carbetapentane, and dextramethorphan; a diuretic; and a sedating or non-sedating antihistamine.

9. The method of claim 7, wherein said additional therapeutic compound is an NSAID.

10. The method of claim 9, wherein said NSAID is a non-selective COX-2 inhibitor.

11. The method of claim 9, wherein said NSAID is a selective COX-2 inhibitor.

12. The method of claim 11, wherein said selective COX-2 inhibitor is selected from the group consisting of rofecoxib, etoricoxib, celecoxib, parecoxib and valdecoxib.

13. The method of claim 1, wherein said PPAR-δ agonist has an EC₅₀ less than 1 μM as measured by the GAL4 transactivation assay.

14. Cancelled

15. The method of claim 1, wherein said PPAR-δ agonist has an IC₅₀ less than 100 nM as measured by the PPAR Scintillation Proximity Assay using Compound B as the standard.

16-17. Cancelled.

18. The method of claim 1, wherein said PPAR-δ agonist has an IC₅₀ for binding PPAR-α that is greater than 1 μM and an IC₅₀ for binding PPAR-γ that is greater than 1 μM, both measured by the PPAR Scintillation Proximity Assay using Compound A as the standard.

19. Cancelled

20. The method of claim 1, wherein said PPAR-δ agonist has an EC₅₀ for PPAR-α agonism that is greater than 500 nM and an EC₅₀ for PPAR-γ agonism that is greater than 1 μM, both as measured by the the GAL4 transactivation assay.

21. Cancelled

22. A pharmaceutical composition which comprises a PPAR-δ agonist, an NSAID and a pharmaceutically acceptable carrier.

23. The pharmaceutical composition of claim 22 wherein the NSAID is a non-selective NSAID.

24. The pharmaceutical composition of claim 22 wherein the NSAID is a selective COX-2 inhibitor.

25. The pharmaceutical composition of claim 22 wherein the selective COX-2 inhibitor is selected from the group consisting of rofecoxib, etoricoxib, celecoxib, parecoxib and valdecoxib.
